SEQUENCE LISTING (15 sequences)

SEQ ID NO: 1 - DNA, 207 bp, Saccharomyces cerevisiae
atgaccatggaccaaggccttaacccaaagcaattcttccttgacgatgtcgtcctacaagacactttgtgctcaatgagcaaccgtgtcaacaagagtgtcaagaccggctacttattccccaaggatcacgttccttctgccaacatcattgccgtcgaacgtcgcggcggtctttctgacattggtaagaatacttccaactaa

SEQ ID NO: 2 - DNA, 65 bp, Artificial
  Synthetic polynucleotide
tatatataactacaaaccacatcagcaataaaaaaaaactatatgcggatccccgggttaattaa

SEQ ID NO: 3 - DNA, 62 bp, Artificial
  Synthetic polynucleotide
ctttctcattggcctacaaaaaaaaagagaagcatgctcttagaattcgagctcgtttaaac

SEQ ID NO: 4 - DNA, 65 bp, Artificial
  Synthetic polynucleotide
ctttcctttcttgttcttttacttataagaaaaataatatacgtatatatataactacaaaccac

SEQ ID NO: 5 - DNA, 65 bp, Artificial
  Synthetic polynucleotide
taaaaacatatctactatattctgacgtatttataatctattgttctttcctatcattggcctac

SEQ ID NO: 6 - DNA, 20 bp, Artificial
  Synthetic polynucleotide
tgccttcgtatctcgtttcc

SEQ ID NO: 7 - DNA, 20 bp, Artificial
  Synthetic polynucleotide
aaagggcagattgtgtggac

SEQ ID NO: 8 - DNA, 65 bp, Artificial
  Synthetic polynucleotide
ttttactgagaatcaagagaagctagagagggattggtctcaatgcggatccccgggttaattaa

SEQ ID NO: 9 - DNA, 65 bp, Artificial
  Synthetic polynucleotide
ttttttcttatataaatgtcagtaatacatgatatctttaagttagaattcgagctcgtttaaac

SEQ ID NO: 10 - DNA, 20 bp, Artificial
  Synthetic polynucleotide
caattgggccgtatatctcg

SEQ ID NO: 11 - DNA, 20 bp, Artificial
  Synthetic polynucleotdie
gatggtcggaagaggcataa

SEQ ID NO: 12 - DNA, 65 bp, Artificial
  Synthetic polynucleotide
ttgacgcaagttgtacctgctcagatccgataaaactcgactatgcggatccccgggttaattaa

SEQ ID NO: 13 - DNA, 65 bp, Artificial
  Synthetic polynucleotide
ggttataaataggatataatataatatagggatcaagtacaactagaattcgagctcgtttaaac

SEQ ID NO: 14 - DNA, 20 bp, Artificial
  Synthetic polynucleotide
acgcaagttgtacctgctca

SEQ ID NO: 15 - DNA, 20 bp, Artificial
  Synthetic polynucleotide
acaaaagagcaaaggctgga

(19) United States
(12) Patent Application Publication
(10) Pub. No.: US 2008/0187927 A1
(43) Pub. Date: Aug. 7, 2008

(54) USING HUG1 PROMOTER AND A REPORTER GENE TO DETECT GENOTOXICITY AND GENOPROTECTION

(76) Inventors: Sean P. Palecek, Verona, WI (US); Michael G. Benton, Prairieville, LA (US)

Correspondence Address: QUARLES & BRADY LLP, 33 E. MAIN ST, SUITE 900, P.O. BOX 2113, MADISON, WI, 53701-2113, US

(21) Appl. No.: 11/938,640

(22) Filed: Nov. 12, 2007

Related U.S. Application Data
(60) Provisional application No. 60/865,435, filed on Nov. 12, 2006.

(51) Int. Cl. C12Q 1/68; C12N 15/00; C12N 1/00

(52) U.S. Cl. 435/6; 435/320.1; 435/255.1; 435/255.2

(57) ABSTRACT

Methods are disclosed for genotoxicity testing and genoprotection testing using genetic constructs having a hydroxyurea- and UV- and gamma radiation-induced promoter linked to a reporter gene, such as green fluorescent protein.

### CROSS-REFERENCE TO RELATED APPLICATIONS

This application claims the benefit of U.S. Provisional Patent Application No. 60/865,435, filed Nov. 12, 2006, incorporated herein by reference as if set forth in its entirety.

### STATEMENT REGARDING FEDERALLY SPONSORED RESEARCH OR DEVELOPMENT

This invention was made with United States government support awarded by the following agency: National Science Foundation Grant No. BES-0238680. The United States government has certain rights in this invention.

### BACKGROUND

The invention relates generally to methods and compositions for detecting genotoxins or putative inhibitors of genotoxins, and more particularly to methods and compositions including yeast-based biosensors having a hydroxyurea- and UV- and gamma radiation-induced (HUG1) promoter linked to a reporter gene, for detecting genotoxins or putative inhibitors of genotoxins.

Genotoxins are chemical compounds capable of damaging genetic material, such as DNA. Genotoxins may act directly by binding to DNA or may act indirectly by affecting enzymes involved in DNA replication. In addition to chemical compounds, radiation is considered a genotoxin. Genotoxins can therefore cause mutations that may or may not lead to cancer or birth defects (inheritable damage).

Typical tests for genotoxins include in vitro and in vivo tests designed to detect compounds that induce genetic damage directly or indirectly by various mechanisms. Of particular interest herein are tests that utilize microorganisms.

Typically, bacteria, which are prokaryotes, are used as biosensors for genotoxicity testing. Traditional prokaryote-based biosensor systems include the Ames test, the umu-test and the SOS-chromotest. Yeast, however, are an attractive alternative to bacteria in genotoxicity testing for several reasons. Most importantly, yeast are eukaryotic. As such, gene regulation and biochemical pathways for responding to DNA damage in yeast are more similar to those of higher organisms than they are to those of prokaryotes. For example, many DNA damage sensory and repair mechanisms in humans have been identified from their homology to similar mechanisms in yeast. In general, yeast are more physically robust than many prokaryotes, so they are viable in a wider variety of testing environments.

U.S. Pat. No. 6,489,099 to Walmsley & Heyer, incorporated herein by reference as if set forth in its entirety, disclosed yeast promoters (e.g., RAD2, RAD6, RAD7, RAD18, RAD23, RAD51, RAD54, CDC7, CDC8, CDC9, MAG1, PHR1, DIN1, DDR48, RNR1, RNR2, RNR3 and UB14) operably linked to a light-emitting reporter gene for identifying agents that cause or potentiate DNA damage. Others, however, have shown that these promoters, including RAD54, are not necessarily sensitive to genotoxins and are not entirely selective to DNA damage caused by genotoxins. In fact, RAD54 responds to glycerol, heat shock and nitrogen deprivation. See, Smith J, et al., “Transcriptome profiling to identify genes involved in peroxisome assembly and function,” J. Cell Biol. 158:259-271 (2002); and Gasch A, et al., “Genomic expression programs in the response of yeast cells to environmental changes,” Mol. Biol. Cell 11:4241-4257 (2000). Moreover, Walmsley & Heyer, and others, did not disclose a eukaryotic-based biosensor that is selective to DNA damage caused by particular genotoxins, that has increased sensitivity (i.e., responds to lower doses and provides a more robust response) to such genotoxins or that has non-light emitting reporter genes.

For the foregoing reasons, there is a need for additional yeast-based biosensors for genotoxicity testing.

### BRIEF SUMMARY

In accord with the present invention, an in vivo, yeast-based system for genotoxicity assays employs a vector active in a yeast host cell, the vector including a detectable reporter gene operably linked to and under control of a promoter that increases transcription in response to DNA damage caused genotoxins, but not in response to environmental conditions (e.g., heat shock, cold shock, osmotic shock and starvation) and non-genotoxic chemical agents that can result in cell damage or death.

In a first aspect, an expression vector for genotoxicity or genoprotection testing is summarized as having a HUG1 promoter (HUG1P; SEQ ID NO:1) operably linked to a reporter gene not natively linked to HUG1P, such that the promoter increases transcription in response to DNA damage caused genotoxins, but not in response to heat shock, starvation or formaldehyde.

In some embodiments of the first aspect, the reporter gene is GFP, luciferase, β-galactosidase or yeast-enhanced green fluorescent protein (yEGFP).

In some embodiments of the first aspect, the genotoxins are alkylating agents, chemotherapeutic agents, DNA cross-linking agents, DNA topoisomerase inhibitors, oxidizing agents, ribonucleotide reductase inhibitors, UV light or ionizing radiation.

In a second aspect, a host cell for genotoxicity testing is summarized as a yeast having the expression vector described above.

In some embodiments of the second aspect, the yeast is Saccharomyces cerevisiae.

In some embodiments of the second aspect, the yeast further lacks one or more gene involved in a cellular response to DNA damage in yeast. For example, the deleted gene can be at least one of MAG1 and MRE11.

In a third aspect, a method for identifying a genotoxic agent is summarized as including the steps of exposing the host cells described above to an agent suspected of being a genotoxin, and then measuring expression of the reporter gene. Genotoxicity of the agent correlates with increased expression of the reporter gene of host cells exposed to the agent relative expression of the reporter gene of host cells not exposed to the agent.

In a related aspect, a method for identifying a genoprotective agent is summarized as including the steps of exposing the host cells described above to a genotoxic agent and an agent suspected of being genoprotective, and then measuring expression of the reporter gene. Genoprotectivity of the agent vis-à-vis the genotoxic agent correlates with no increase or a decrease in expression of the reporter gene in host cells exposed to both agents relative to expression of the reporter gene of host cells exposed only to the genotoxic agent.

These and other features, aspects and advantages of the present invention will become better understood from the description that follows. The present invention is not intended to be limited to the foregoing, but rather to encompass all such variations and modifications as come within the scope of the appended claims. Reference should therefore be made to the claims herein for interpreting the scope of the invention.

### BRIEF DESCRIPTION OF THE DRAWINGS

Not applicable.

### DESCRIPTION OF PREFERRED EMBODIMENTS

The present invention relates to the inventors observation that the gene HUG1 is sensitive to genotoxins, even though its precise molecular function remains unknown. This observation suggests that the HUG1 promoter (HUG1P) may be operably linked to a reporter gene to identify genotoxic agents or agents suspected of being genoprotective.

The inventors describe herein a HUG1P-GFP promoter-reporter construct effective for detecting genotoxicity. While the exact biological function of HUG1 remains a mystery, it is known to be involved after DNA damage in the Mec1p kinase signal transduction pathway. Basrai M, et al., “NORF5/HUG1 is a component of the MEC1-mediated checkpoint response to DNA damage and replication arrest in Saccharomyces cerevisiae,” Mol. Cell. Biol. 19:7041-7049 (1999). The inventors discovered that HUG1P exhibited a dose-dependent transcriptional response to genotoxins, such as a DNA alkylating agent, methyl methanesulfonate and gamma radiation (γ-ray). Thus, HUG1P appears useful for regulating reporter gene expression in response to DNA damage; however, optimal genotoxicity detection requires enhancing the host cell's sensitivity and selectivity to specific types of DNA damage.

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are described herein.

In describing the embodiments and claiming the invention, the following terminology will be used in accordance with the definitions set out below.

As used herein:, “genotoxin” means a chemical or other agent that causes damage or mutation to DNA. Exemplary genotoxins include, but are not limited to, alkylating agents (e.g., agents that react chemically with DNA and disrupt it in the S-phase, such as nitrogen mustards, ethylenimes, alkylsulfonates, triazenes, piperazines and nitrosureas), antibiotics (e.g., agents that form a complex with DNA and interfere with DNA, RNA and protein synthesis, such as anthracyclines, dactinomycin, bleomycin, adriamycin and mithramycin), microtubule inhibitors (e.g., agents that inhibit depolymerization of tubulin, such as taxol, vinblastine and vincristine), DNA cross-linking agents (e.g., agents that, cause intrastrand DNA crosslinks, interstrand DNA crosslinks or crosslinks between DNA and protein, such as carboplatin, cisplatin and mitomycin C), DNA topoisomerase inhibitors (e.g., agents that inhibit the dissociation of topoisomerase and DNA, leading to replication-mediated DNA damage, or agents that induce single- or double-stranded breaks in DNA, such as camptothecins and etoposide), hydroxyurea and other ribonucleotide reductase inhibitors (e.g., translation inhibitors, dimerization inhibitors and catalytic inhibitors), oxidizing agents (e.g., agents that cause nucleotide radicals, such as lipid peroxidation products and hydrogen peroxide), UV light and ionizing radiation (e.g., γ-ray).

As used herein, “genoprotective” means that an agent attenuates or prevents DNA damage caused by genotoxins. In general, an agent is genoprotective if it reduces expression of the reporter gene incorporated into the expression sequences disclosed herein in cells exposed to the genoprotective agent and a genotoxin by at least 1%, alternatively by at least 2.5%, alternatively by at least 5%, alternatively by at least 10% aiid alternatively by at least 15% when compared to cells exposed only to the genotoxin.

As used herein, a “coding sequence” means a sequence that encodes a particular polypeptide, and is a nucleic acid sequence that is transcribed (in the case of DNA) and translated (in the case of mRNA) into a polypeptide in vitro or in vivo when placed under the control of appropriate regulatory sequences. The boundaries of the coding sequence are determined by a start codon at a 5′ (amino) terminus and a translation stop codon at a 3′ (carboxy) terminus. A coding sequence can include, but is not limited to, viral nucleic acid sequences, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, “control sequences” means promoter sequences, polyadenylation signals, transcription termination sequences, upstream regulatory domains, origins of replication, internal ribosome entry sites (“IRES”), enhancers, and the like, which collectively provide for replication, transcription and translation of a coding sequence in a recipient cell. Not all of these control sequences need always be present so long as the selected coding sequence is capable of being replicated, transcribed and translated in an appropriate host cell.

As used herein, an “expression sequence” means a control sequence operably linked to a coding sequence.

As used herein, a “promoter” means a nucleotide region comprising a nucleic acid (i.e., DNA) regulatory sequence, wherein the regulatory sequence is derived from a gene that is capable of initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is repressed by an analyte, cofactor, regulatory protein, etc.) and “constitutive promoters” (where expression of a polynucleotide sequence operably-linked to the promoter is unregulated and therefore continuous).

As used herein, “operably linked” means that elements of an expression sequence are configured so as to perform their usual function. Thus, control sequences (i.e., promoters) operably linked to a coding sequence are capable of effecting expression of the coding sequence. The control sequences need not be contiguous with the coding sequence, so long as they function to direct the expression thereof. Thus, for example, intervening untranslated, yet transcribed, sequences can be present between the promoter and the coding sequence, and the promoter sequence can still be considered “operably linked” to the coding sequence.

As used herein, a “vector” means a replicon, such as a plasmid, phage or cosmid, to which an exogenous polynucleotide segment is incorporated so as to bring about the replication of the incorporated polynucleotide segment. A vector is capable of transferring gene sequences to a host cell and may be non-integrated (i.e., self-replicating) or integrated into the host cell's genome.

Typically, the terms “vector construct,” “expression vector,” “gene expression vector,” “gene delivery vector,” “gene transfer vector” and “cassette” all refer to an assembly that is capable of directing the expression of a sequence or gene of interest. Thus, the terms also include cloning and expression vehicles.

As used herein, “isolated polynucleotide” o r “isolated polypeptide” means a polynucleotide or polypeptide removed from its natural environment or prepared using synthetic methods, such as those known to one of ordinary skill in the art. The polynucleotides and polypeptides described herein can purified from normally associated material in conventional ways, such that in the purified preparation the polynucleotide or polypeptide is the predominant species in the preparation. Complete purification is not required. At the very least, the degree of purification is such that extraneous material in the preparation does not interfere with use of the polynucleotide or polypeptide in the manner disclosed herein. The polynucleotide or polypeptide is at least about 85% pure; alternatively at least about 95% pure; and alternatively, at least about 99% pure.

Further, an isolated polynucleotide has a structure that is not identical to that of any naturally occurring nucleic acid molecule or to that of any fragment of a naturally occurring genomic nucleic acid spanning more than one gene. An isolated polynucleotide also includes, without limitation, (a) a polynucleotide having a sequence of a naturally occurring genomic or extra-chromosomal nucleic acid molecule, but which is not flanked by the coding sequences that flank the sequence in its natural position; (b) a polynucleotide incorporated into a vector or into a prokaryote or eukaryote host cell's genome such that the resulting polynucleotide is not identical to any naturally occurring vector or genomic DNA; (c) a separate molecule such as a cDNA, a genomic, fragment, a fragment produced by polymerase chain reaction (PCR) or a restriction fragment; and (d) a recombinant polynucleotide sequence that is part of a hybrid gene (i.e., a gene encoding a fusion polypeptide). Specifically excluded from this definition are nucleic acids present in mixtures of clones, e.g., as these occur in a DNA library such as a cDNA or genomic DNA library. An isolated polynucleotide can be modified or unmodified DNA or RNA, whether fully or partially single-stranded, double-stranded or even triple-stranded. In addition, an isolated polynucleotide can be chemically or enzymatically modified and can include so-called non-standard bases such as inosine.

As used herein, “homologous” refers those polynucleotides sharing at least 90% or at least 95% sequence identity to SEQ ID NO:1 that fuctions as a promoter. For example, a polynucleotide that is at least 90% or at least 95% identical to SEQ ID NO:1 is expected to be a DNA damage-sensitive promoter. One of ordinary skill in the art understands that modifications to either the polynucleotide includes substitutions, insertions (e.g., adding no more than ten nucleotides) and deletions (e.g., deleting no more than ten nucleotides). These modifications can be introduced into the polynucleotides described below without abolishing structure and ultimately, function. Polynucleotides containing such modifications can be used in the methods of the present invention. Such polynucleotides can be identified by using the screening methods described below.

An isolated polynucleotide (or its complement) that can hybridize to any of the uninterrupted polynucleotide sequences described herein, under either stringent or moderately stringent hybridization conditions, is also within the scope of the present invention. Stringent hybridization conditions are defined as hybridizing at 68° C. in 5×SSC/5×Denhardt's solution/1.0% SDS, and washing in 0.2×SSC/0.1% SDS±100 μg/ml denatured salmon sperm DNA at room temperature (RT), and moderately stringent hybridization conditions are defined as washing in the same buffer at 42° C. Additional guidance regarding such conditions is readily available in the art, e.g., in Sambrook et al., Molecular Cloning, A Laboratory Manual (Cold Spring Harbor Press, N.Y. 1989); and Ausubel et al. (eds.), Current Protocols in Molecular Biology, Unit 2.10 (John Wiley & Sons, N.Y. 1995).

It is well known to one of ordinary skill in the art that amino acids within the same conservative group can typically substitute for one another without substantially affecting the function of a polypeptide. For the purpose of the present invention, such conservative groups are set forth in Table 1 and are based on shared properties.

TABLE 1
Amino Acid Conservative Substitutions.

| Original Residue | Conservative Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala |

Reporter genes permit detection of expression from the expression sequences described herein, and therefore allow one to monitor whether an agent is genotoxic or not. One reporter gene useful with the expression sequences described herein is GFP or its derivatives. GFP is isolated from the jellyfish Aequorea victoria/Aequorea aequorea/Aequorea forskalea and fluoresces green when exposed to blue light. Prendergast F & Mann K, “Chemical and physical properties of aequorin and the green fluorescent protein isolated from Aequorea forskalea,” Biochemistry 17:3448-3453 (1978), incorporated herein by reference as if set forth in its entirety. GFP may be advantageously used as a reporter gene because the protein is simple to measure, requires no additional reagents for detection and is non-toxic.

Derivatives of GFP include polynucleotide sequences that encode polypeptide analogues or polypeptide fragments of GFP that are able to emit light. Many of these derivatives absorb and re-emit light at wavelengths different than GFP found endogenously in A. victoria. For example, preferred polynucleotides encode a S65T derivative of GFP (in which serine 65 of GFP is replaced by a threonine). GFP-S65T, which was used in the Examples below, has an advantage in that it photobleaches slower than, and is brighter than, wild-type GFP (when excited at its longest-wavelength peak). A commercially available vector harboring GFP-S65T can be used with the methods described below (pFA6a-GFP(S65T)-His3MX6, described in more detail below).

A second reporter gene useful with the expression sequences described herein is luciferase. This enzyme used in nature for bioluminescence. The most widely used luciferase is from the firefly Photinus pyralis. Baldwin T, “Firefly luciferase: the structure is known, but the mystery remains,” Structure 4:223-228 (1996), incorporated herein by reference as if set forth in its entirety). A commercially available vector harboring luciferase is available and can be used with the methods described herein (pFR-Luc; Stratagene; La Jolla, Calif.).

A third reporter gene useful with the expression sequences described herein is β-galactosidase. This enzyme catalyzes the hydrolysis of β-galactoside into monosaccharides and causes an organism expressing the gene to appear blue when grown on a medium that contains the substrate analog X-gal. In some instances, an inducer molecule, such as isopropyl β-D-1-thiogalactopyranoside (IPTG), is needed when the genesis under control of its native promoter. A commercially available vector harboring β-galactosidase is available and could be used with the methods described below (pCMVβ; Clontech Laboratories, Inc.; Mountain View, Calif.).

A fourth reporter gene useful with the expression sequences described herein is yEGFP. yEGFP has an amino acid sequence adapted for usage in yeast. Cormack B., et al., “Yeast-enhanced green fluorescent protein (yEGFP) a reporter of gene expression in Candida albicans,” Microbiology 143:303-311 (1997), incorporated herein by reference as if set forth in its entirety. That is, some yeast use an alternative codon system in which a CUG codon for leucine is read as serine. Thus, yEGFP is particularly suitable for detecting gene expression in yeast. Likewise, polynucleotides coding yEGFP are also useful because yEGFP is less heat sensitive than nascent GFP. Moreover, yEGFP shows increased sensitivity compared to nascent GFP. Billinton N, et al., “Development of a green fluorescent protein reporter for a yeast genotoxicity biosensor,” Biosens. Bioelectron. 13:831-8 (1998), incorporated herein by reference as if set forth in its entirety; see also, Walmsley & Heyer, supra.

The expression sequences described herein are suitable for incorporation into a vector. As noted above, such vectors include plasmids, phages and cosmids. Each of these classes of vectors is well known to one of ordinary skill in the art, and therefore need not be described in detail. However, the vectors should include one or more selectable markers to enable selection of host cells containing the vector. For example, the vectors can include a selectable marker such as a gene conferring resistance to kanamycin (or G148) or ampicillin. Alternatively, selectable markers can include a gene that restores prototrophy, such as the yeast URA3 gene.

Vectors can be designed so that the vector is non-integrating (i.e., replicative) into the host cell's genome, but rather autonomously replicates in the cytosol of the host cell. That is, elements needed for vector replication are in the vector itself. These replicating vectors can give rise to multiple copies of the DNA molecule in a transformant, and are therefore useful when over-expression of the reporter gene is desired.

Alternatively, vectors may be designed so that the vector integrates into the host cell's genome. An integrating vector has improved stability compared to a non-integrating vector. Integrating vectors have DNA sequences that facilitate homologous recombination. For example, incorporating fragments of the HO gene from chromosome IV of S. cerevisiae into the vector allows targeted integration into the genome of S. cerevisiae or cell-lines derived therefrom. Preferably, the vectors may be formed from pFA vectors or derivatives thereof that are known to one of ordinary skill in the art. See, Wach A, et al., “Heterologous HIS3 marker and GFP reporter modules for PCR-targeting in Saccharomyces cerevisiae,” Yeast 13:1065-1075 (1997), incorporated herein by reference as if set forth in its entirety. Moreover, a vectors should permit site-specific integration of a reporter at a correct location and should contain a selectable marker to identify transformed cells. Preferable selectable markers include, but are not limited to, HIS3, LEU2, ARG4 and URA3. It is also contemplated that any of the various antibiotic resistance genes known to one of ordinary skill in the art would be acceptable to identify transformed cells.

Exemplary vectors are pFA6a-GFP(S65T)-His3MX6 and pFA6a-GFP(S65T)-KanMX6, which are used in the Examples below. These plasmids contain GFP-S65T and either HIS3 or KAN as the selectable marker, respectively.

Although stably transformed host cells are preferred, the use of transiently transformed host cells is not precluded.

Suitable host cells for the methods described herein include yeasts. Yeast can be easily manipulated like prokaryotes, but are eukaryotic and have DNA repair systems more closely related to humans than to those of prokaryotes. As such, methods for detecting DNA damage are advantageously conduced in yeast, as compared to the Ames tests the umu-test and the SOS-chromotest.

Any yeast strain would be suitable for use with the vectors and methods described herein. Suitable yeast include, but are not limited to, S. cerevisiae, including such strains as S288C, W303, D273-10B, X2180, A364A, Σ1278B, AB972, SK1 and FL100 (many of which are available at ATCC (Manassas, Va.) or the National Collection of Yeast Cultures (Colney, Norwich, United Kingdom)).

Yeast can contain other modifications that influence the sensitivity and type of DNA damage response detected. In this regard, yeast can be made more selective and more sensitive to certain genotoxins if they lack genes involved in DNA damage responses, DNA repair mechanisms, ribonucleotide synthesis, cell wall structure and efflux pumps. Such modifications reduce the cell's ability to repair its DNA and its ability to serve as a DNA damage-sensitive biosensor.

One gene that can be deleted to enhance sensitivity is MRE11, a gene that plays a role in non-homologous end-joining (NHEJ) DNA double-strand break (DSB) repair pathways and is part of the highly conserved RMX complex that aids in sensing and repairing double strand breaks in budding yeasts. Another gene that can be deleted to enhance sensitivity is MAG1, a gene that encodes a DNA glycosylase that is a member of the base-excision repair (BER) pathway. As shown below, yeast lacking MRE11 and/or MAG1 showed increased sensitivity to alkylating agents and radiation.

The methods described herein are suitable for assessing whether and to the extent that a genotoxin cause DNA damage. As such, the methods are useful for identifying an agent that causes DNA damage. In addition, the methods are useful for assessing whether it is safe to expose an organism, such as a human, to a particular level of a genotoxin. For example, the methods may be used as a mutagenesis assay for screening whether a suspected genotoxin induces DNA damage. Alternatively, the methods may be used to monitor whether a test sample (e.g., soil or water sample) is contaminated with a genotoxin.

Likewise, the methods described herein are suitable for assessing whether and to what extent an agent may protect against DNA damage (i.e., genoprotective). As such, the methods are useful for identifying agents that either can interfere directly with a genotoxin or can interact with an area of DNA that the genotoxin acts upon.

The invention will be more fully understood upon consideration of the following non-limiting Examples.

### EXAMPLES

### Example 1

### Yeast-Based Biosensors Having HUG1P-GFP

Yeast having a HUG1-GFP promoter/reporter construct (called SPY1101; genotype is W303 MA:Ta/a ura3-52/ura3-52 his3::hisG/his3::hisG leu2::hisG/leu2::hisG HUG1P-GFP::HIS3/HUG1P-HUG1) were derived from S. cerevisiae strain SPY810 (wild-type yeast; genotype is W303 MATa/a ura3-52/ura3-52 his3::hisG/his3::hisG/leu2::hisG/leu2::hisG) using a PCR-based gene deletion method. Longtine M, et al., “Additional modules for versatile and economical PCR-based gene deletion and modification in Saccharomyces cerevisiae,” Yeast 14:953-961 (1998), incorporated herein by reference as if set forth in its entirety. Briefly, plasmid pFA6a-GFP(S65T)-His3MX6 (kindly provided by John Pringle at The University of North Carolina at Chapel Hill (Chapel Hill, N.C.); see also, Longtine et al., supra.), which contains GFP-S65T, was isolated from Escherichia coli DHα with a QIAprep Spin Miniprep Kit (Qiagen; Santa Clarita, Calif.).

50 μM HUG1 forward deletion primer 1 (SEQ ID NO:2) and 50 μM HUG1 reverse deletion primer 1 (SEQ ID NO:3) were used in a PCR reaction with about 100 μg pFA6a-GFP(S65T)-His3MX6 to create a cassette that included a fusion polynucleotide encoding GFP-S65T and HIS3 (a gene that encodes an enzyme for catalyzing the sixth step in histidine biosynthesis) flanked by 45 bp regions homologous to a 45 bp-region upstream and downstream from the HUG1 ORF. Then, 50 μM HUG1 forward deletion primer 2 (SEQ ID NO:4) and 50 μM HUG1 reverse deletion primer 2 (SEQ ID NO:5) were used with about 100 μg of the cassette as a template to add another 45 bp to the flanking region, such that the cassette included the GFP-HIS3 fusion flanked by 90 bp regions homologous to a 90 bp upstream (including HUG1P) and downstream from the HUG1 ORF. PCR reactions were performed using Pfx enzyme with 1 cycle at 94° C. for 5 minutes, followed by 34 cycles at 94° C. for 30 seconds, 48° C. for 30 seconds, 68° C. for 2.5 minutes, followed by 1 cycle at 68° C. for 5 minutes.

Wild-type yeast (SPY810) were, stably transformed with the cassette that included the GFP-HIS3 fusion flanked by 90 bp regions homologous to the 90 bp upstream and downstream from the HUG1 ORE using a lithium acetate protocol as described by Gietz et al. Gietz D, et al., “Improved method for high efficiency transformation of intact yeast cells,” Nucleic Acids Res. 20:1425 (1992), incorporated herein by reference as if set forth in its entirety. Transformants were selected on synthetic complete (SC) medium lacking histidine. See, Sherman F, “Getting started with yeast,” Methods Enzymol. 350:3-41 (2002), incorporated herein by reference as if set forth in its entirety. Transformants were then verified by performing PCR, using a primer (SEQ ID NO:6) approximately 500 bp upstream from the HUG1 start site and a primer (SEQ ID NO:7) within the GFP coding region. PCR reactions were performed using Taq enzyme with 1 cycle at 94° C. for 5 minutes, followed by 34 cycles at 94° C. for 30 seconds, 55° C. for 30 seconds, 72° C. for 2.5 minutes, followed by 1 cycle at 72° C. for 5 minutes. SPY1101 produced DNA fragments approximately 856 bp long, while untransformed cells produced no DNA fragment.

### Example 2

### Genotoxins Induce Measurable GFP Expression in the Yeast-Based Biosensor

Methods:

Yeast from Example 1 (SPY1101) were exposed to the genotoxins shown in Table 2, which included alkylating agents, oxidizing agents, ribonucleotide reductase inhibitors, UV mimetic agents, glycopeptide antibiotics, quinoline-based alkaloids and ionizing radiation. Each genotoxin was added directly to a culture of SPY1101. Fresh phleomycin (Sigma) was aliquoted in water at a concentration of 20 mg/ml and stored at −20° C. until culture exposure. Likewise, CPT (Sigma) was aliquoted in dimethylsulfoxide (DMSO) at a concentration of 10 mg/ml and stored at −20° C. until culture exposure. All samples exposed to CPT were adjusted with DMSO, such that the final concentration of DMSO in the culture was 1% (v/v), regardless of CPT dose. γ-ray exposure was done using a Cs¹³⁷ source (exposure was 7 Gy/min and ended when the desired dose was reached). For all genotoxins (except γ-ray), exposure was continuous.

All DNA damage experiments included a minimum of three independent trials. SPY1101 were grown in rich medium (see, Sherman, supra) at 30° C. and with gentle shaking at 250 rpm to mid log-phase and then diluted to an OD₆₀₀ of 0.1. After dilution, the culture was divided into 10 ml aliquots for exposure to one of the DNA damaging agents listed above. One culture aliquot was treated as a control and received no genotoxin, while the other aliquots each received one of seven different doses of the genotoxin, as listed in Table 2. In addition, SPY810 (wild-type control) were similarly treated. All cultures were then returned to 30° C. for incubation.

TABLE 2
Representative Genotoxins and Dose Ranges.

| Genotoxin | Dose Ranges | |
|---|---|---|
| methyl methanesulfonate (MMS; Sigma-Aldrich; | 0.0005-0.2% | |
| St. Louis, MO) | | |
| ethyl methanesulfonate (EMS; Sigma-Aldrich) | 0.005-2% | |
| 1,2-dimethyl hydrazine (SDMH; Sigma-Aldrich) | 0.005-2% | |
| hydrogen peroxide (H₂O₂; Sigma-Aldrich) | 0.001-0.6 | mM |
| hydroxyurea (HU; Sigma-Aldrich) | 0.1-100 | mM |
| 4-nitroquinoline-1-oxide (4-NQO; Sigma-Aldrich) | 0.001-0.3 | μg/ml |
| Phleomycin (Sigma-Aldrich) | 0.005-4 | μg/ml |
| camptothecin (CPT; Sigma-Aldrich) | 0.01-15 | μg/ml |
| γ-ray | 0.5-200 | Gy |

One hour after exposure, 1 ml of each culture was removed, and the OD₆₀₀ was recorded to monitor culture growth. The cells were washed in PBS, re-suspended in fresh PBS to an OD₆₀₀ of 0.2 and then placed in the dark on ice until flow cytometry analysis. This process was repeated every hour until eight hours had passed since the initial genotoxin exposure. The process was also repeated at twenty-four hours after initial genotoxin exposure.

Flow cytometry analysis was conducted as follows. For each replicate, at least 10,000 cells were analyzed for fluorescence using a flow cytometer (Becton Dickinson; Franklin Lakes, N.J.), and GFP induction analysis was performed with CellQuest™ and Flow-Jo software. Surviving cells were defined as whole cells (measured with light scattering) with intact cellular membranes (measured by exclusion of propidium iodide). All surviving cells were gated and used to measure GFP expression of the population. GFP expression histograms were prepared with WinMDI (available on the world wide web), using a live/dead gate based on propidium iodide exclusion. Histograms were created using a smoothing setting of 5. To eliminate false-positive results because of auto-fluorescence of dead cells or residual medium, both a GFP fold induction of greater than 2 and a p-value less than or equal to 0.05 (by comparing exposed cells to non-exposed cells) were required to conclude that GFP was significantly up-regulated by a genotoxin.

Growth rate analysis was performed as follows. Culture OD₆₀₀ was monitored as a function of exposure time to determine the effect of DNA damaging agent exposure on cell proliferation and doubling time. Two hours after exposure began, 1 ml of each culture was removed, and the OD₆₀₀ was recorded. The OD₆₀₀ was converted to cell titer using a standard curve constructed by measuring the OD₆₀₀ of samples whose cell concentration was determined by counting cells on a hemocytometer. Culture doubling time was calculated using the following equation:

N=2^{t/td}Nₒ; where N is the number of cells at time t, Nₒ is the initial number of cells, and t_{d} is the average doubling time.

Fluorescence microscopy was performed as follows. Microscopic images were acquired using an Olympus® IX70 inverted epifluorescence microscope (Leeds Precision Instruments; Minneapolis, Minn.) with a 40× objective and a GFP filter cube. Bright-field images were used to focus on the cells, and a Nikon® spot camera captured 4 second-exposures to a 100 W mercury lamp. MetaVue 5.0r1 Imaging Software (Molecular Devices Corporation; Downingtown, Pa.) was used to control the camera and the image acquisition. Yeast for microscopic imaging were fixed to a microscope slide.

Results:

SPY1101 expressed GFP in a dose-dependent manner in response to all tested genotoxins. Moreover, all tested genotoxins induced expression of GFP for at least one of the doses tested, indicating that one could expect a detectable level of GFP expression upon exposure of SPY1101 to distinct classes of DNA damaging agents.

Optimum exposure time, and the time that yielded maximum GFP induction, varied among the genotoxins. For several tested genotoxins, (e.g., MMS, EMS, SDMH, phleomycin and CPT), longer exposure led to higher GFP fluorescence. HU, however, induced maximum fluorescence at six to eight hours post-exposure. Likewise, 4-NQO, H₂O₂ and γ-ray resulted in peak fluorescence at less than eight hours (six, four and three hours, respectively). Although no single ideal time point was identified for monitoring GFP induction in SPY1101, around six to eight hours provided an adequate dynamic range for a wide variety of the genotoxins. In general, the genotoxins affected the total population fluorescence in SPY1101, through both the number of cells expressing GFP and the quantity of GFP in those cells.

To determine the sensitivity of HUG1P-GFP to the genotoxins, the above experiments were repeated, and SPY1101 were exposed to two representative genotoxins, each for an eight hour period—MMS and γ-ray (i.e., 0.0005% and 50 Gy, respectively). These doses meet the criteria of at least two-fold induction with a p-value less than or equal to 0.05, as compared to untreated cells. 0.0005% MMS increased fluorescence 2.4-fold after 6 hours, whereas 50 Gy of γ-ray increased fluorescence 2.9-fold after 1 hour.

To determine the minimum: induction time of HUG1P-GFP to the genotoxins, the above experiments were repeated, and SPY1101 were exposed to MMS and γ-ray at doses that induced the highest level of GFP induction for two hours (0.01% MMS and 200 Gy γ-ray, respectively). 0.01% MMS increased fluorescence by 3-fold after 75 minutes; whereas 200 Gy of γ-ray increased fluorescence 2.4-fold 15 minutes after exposure ended (i.e., 45 minutes after exposure began). SPY810 (wild type control) exposed to 0.01% MMS for six hours did not fluoresce. Likewise, SPY1101 not exposed to MMS (HUG1P-GFP control) did not fluoresce.

### Example 3

### General Cell Stress Did Not Induce GFP Expression in the Yeast-Based Biosensor

Methods:

SPY1101 were also exposed to general cell stress. All general cell stress experiments consisted of a minimum of three independent trials. SPY1101 were grown in rich medium at 30° C. and with gentle shaking at 250 rpm to mid log-phase and then diluted to an OD₆₀₀ of 0.1. After dilution, the culture was divided into aliquots. One culture aliquot was treated as a control, while the others were exposed to the following: (1) exposure to HCl, (2) exposure to elevated temperatures to induce heat shock, or (3) exposure to glucose starvation, as listed in Table 3. In addition, SPY810 (wild-type control) were similarly treated. Samples were taken every hour from each culture and prepared for flow cytometry was described above.

TABLE 3
Representative General Cell Stresses and Conditions.

| Cell Stress | Conditions |
|---|---|
| | 1, 5, 10 or 1.5% 1 N HCl |
| Heat Shock | 30, 40, 50 or 60° C. |
| Glucose Starvation | Cultured with 0, 25, 50, 75 or 100% glucose |

Results:

When exposed to 1, 5, 10 or 15% (v/v) 1 N HCl, the maximum induction of GFP fluorescence was 1.5 fold, below the established positive result threshold. Similar results were observed when SPY1101 we re exposed to heat shock, where the maximum GFP induction was 1.8 fold: after 2 hours at 50° C. Similar results were observed in SPY810. Likewise, when all glucose was removed from log-phase cells for twenty-four hours, the maximum GFP induction was 1.4 fold after 7 hours. Similar results were observed in SPY810. Thus, the HUG1P activity was not responsive to general stress response pathways that are not associated with DNA damage.

### Example 4

### Formaldehyde Did Not Induce Measurable GFP Expression in the Yeast-Based Biosensors

Methods:

SPY1101 were also exposed to formaldehyde (Fisher Biotech; Pittsburgh, Pa.). SPY1101 were grown in rich medium at 30° C. and with gentle shaking at 250 rpm to mid log-phase and then diluted to an OD₆₀₀ of 0.1. After dilution, the culture was divided into aliquots. One aliquot was treated as a control, while the others were exposed to formaldehyde. In addition, SPY810 (wild type controls) were similarly treated. Samples were taken every hour from each culture and prepared for flow cytometry as described above.

Results:

No dose-dependent induction of GFP expression resulted from exposure of HUG1P-GFP cells to formaldehyde. Thus, the HUG1P activity was not responsive to cytotoxicity in the absence of DNA damage.

### Example 5

### Deletion of MAG1 and MRE11 Enhances the Sensitivity of HUG1P-GFP Cassette to Genotoxicity

Methods:

Yeast having a MAG1 deletion (SPY1102; also called mag1Δ) were created from SPY1101. Briefly, plasmid pFA6a-GFP(S65T)-KanMX6 (see, Longtine et al., supra), which contains GFP-S65T, was isolated from E. coli DHα using a QIAprep Spin Miniprep Kit. Then, 50 μM MAG1 forward deletion primer 1 (SEQ ID NO:8) and 50 μM MAG1 reverse deletion primer 1 (SEQ ID NO:9) were used in a PCR reaction with 100 ug of pFA6a-GFP(S65T)-KanMX6 to create a cassette that included a fusion polynucleotide encoding kanamycin resistance flanked by 45 bp regions homologous to a 45 bp region upstream and downstream from the MAG1 ORF. PCR reactions were performed using Pfx enzyme with 1 cycle at 94° C. for 5 minutes, followed by thirty-four cycles at 94° C. for 30 seconds, 48° C. for 30 seconds, 68° C. for 2.5 minutes, followed by 1 cycle at 68° C. for 5 minutes.

SPY1101 were stably transformed with the cassette using the lithium acetate protocol described above. Transformants were selected on Yeast Extract/Peptone/Dextrose (YPD; see Sherman, supra) medium containing G418 (an aminoglycoside; Clontech Laboratories, Inc.). Transformants were then verified by performing PCR, using a primer approximately 500 bp upstream from the MAG1 start site (SEQ ID NO:10) and a primer within the kanamycin resistance coding region (SEQ ID NO:11). PCR reactions were performed using Taq enzyme with 1 cycle at 94° C. for 5 minutes, followed by thirty-four cycles at 94° C. for 30 seconds, 55° C. for 30 seconds, 72° C. for 2.5 minutes, followed by 1 cycle at 72° C. for 5 minutes. SPY1102 produced DNA fragments approximately 900 bp long, while untransformed cells produced no DNA fragment.

Yeast having a MRE11 deletion (SPY1103; also called mre11ₐΔ) were created from SPY1101. Briefly, plasmid pFA6a-GFP(S65T)-KanMX6 was isolated from E. coli DHα using a Qiaprep Spin Miniprep Kit. Then, MRE11 forward deletion primer (SEQ ID NO:12) and MRE11 reverse deletion primer (SEQ ID NO:13) were used in a PCR reaction with pFA6a-GFP(S65T)-KanMX6 to create a cassette that included a fusion polynucleotide encoding kanamycin resistance flanked by 45 bp regions homologous to the 45 bp region upstream and downstream from the MRE11 ORF. PCR reactions were performed using Pfx enzyme with 1 cycle at 94° C. for 5 minutes, followed by thirty-four cycles at 94° C. for 30 seconds, 48° C. for 30 seconds, 68° C. for 2.5 minutes, followed by 1 cycle at 68° C. for 5 minutes.

SPY1101 were stably transformed with the cassette using the lithium acetate protocol described above. Transformants were selected on Yeast Extract/Peptone/Dextrose (YPD) medium containing G418. Transformants were then verified by performing PCR, using a primer approximately 500 bp upstream from the MRE11 start site (SEQ ID NO:14) and a primer approximately 500 bp downstream from the stop codon (SEQ ID NO:15). PCR reactions were performed using Taq enzyme with 1 cycle at 94° C. for 5 minutes, followed by thirty-four cycles at 94° C. for 30 seconds, 55° C. for 30 seconds, 72° C. for 2.5 minutes, followed by 1 cycle at 72° C. for 5 minutes. SPY1103 produced DNA fragments approximately 1.7 kb long, while: SPY1101 produced DNA fragments approximately 2.2 kb long.

Additional yeast having a MRE11 deletion (SPY1104; also called mre11_{b}Δ) were created from SPY823 (genotype is W303 MATαleu2-3, 112 trp-1 can1-100 ura3-1 ade2-1 his3-11,15 ura3-52 his3::hisG). SPY1103 differs from SPY1104 in that it lacks HUG1P-GFP. Briefly, plasmid pFA6a-GFP(S65T)-KanMX6 was isolated from E. coli DHα using a Qiaprep Spin Miniprep Kit. Then, 50 μM MRE11 forward deletion primer (SEQ ID NO:12) and 50 μM, MRE11 reverse deletion primer (SEQ ID NO:13) were used in a PCR reaction with 100 μg pFA6a-GFP(S65T)-KanMX6 to create a cassette that included a fusion polynucleotide encoding kanamycin resistance flanked by 45 bp regions homologous to the 45 bp region upstream and downstream from the MRE11 ORF. PCR reactions were performed using Pfx enzyme with 1 cycle at 94° C. for 5 minutes, followed by thirty-four cycles at 94° C. for 3.0 seconds, 48° C for. 30 seconds, 68° C. for 2.5 minutes, followed by 1 cycle at 68° C. for 5 minutes.

SPY823 were transformed with the cassette using the lithium acetate protocol described above. Transformants were selected on Yeast Extract/Peptone/Dextrose (YPD) medium containing G418. Transformants were then verified by performing PCR, using a primer approximately 500 bp upstream from the gene MRE11 start site (SEQ ID NO:14) and a primer approximately 500 bp downstream from the stop codon (SEQ ID NO:15). PCR reactions were performed using Taq enzyme with 1 cycle at 94° C. for 5 minutes, followed by thirty-four cycles at 94° C. for 30 seconds, 55° C. for 30 seconds, 72° C. for 2.5 minutes, followed by 1 cycle at 72° C. for 5 minutes. SPY1104 produced DNA fragments approximately 1.7 kb long, while SPY823 produced DNA fragments approximately 2.2 kb long.

Finally, yeast having both a MAG1 and a MRE11 deletion (SPY1105; also called mag1Δ/mre11_{b}Δ) were created from a cross of SPY1102 and SPY1104, which were mated on GNA medium (5% glucose, 3% Difco nutrient broth, 1% yeast extract and 2% agar). Successfully mated cells were selected on SC medium lacking histidine and leucine, grown again on GNA medium, and then sporulated on NGS medium (1% potassium acetate, amino acids and 2% agar) for 7 days to induce them back to a haploid state. Individual haploid cells were isolated by random spore analysis. The resulting strains were screened on SC medium lacking histidine and on YPD medium containing G418. Strains that passed both screenings were analyzed by PCR for MAG1 and MRE11 deletions, as described above. A LEU2 deletion was verified by observing no growth on SC medium lacking leucine. Mating type and ploidy were verified by a mating cross with SKY2164 (MATa ura4) and SKY2165 (MATα ura4) on SC medium lacking uracil. Strains containing these mutations are available from ATCC.

Results:

The cells were exposed to the genotoxins (i.e., MMS, EMS, SDMH, H2O2, HU, 4-NQO, formaldehyde, phleomycin, CPT and γ-ray) as described above. Briefly, two hours after exposure, 1 ml of each culture-was removed and the OD₆₀₀ was recorded to monitor cell proliferation. The cells were then washed in PBS and re-suspended to an OD₆₀₀ of 0.2, and placed in the dark on ice until flow cytometry analysis. This process was repeated after 4, 6 and 8 hours after exposure. The results for MMS and γ-ray are summarized below in Table 4.

TABLE 4
Effect of MAG1 deletion and MRE11 deletion on MMS and γ-ray
sensitivity.

| | | | Relative | | |
|---|---|---|---|---|---|
| Strain | Genotoxin | Dose | Fluorescence | p-value | Exposure (h) |
| SPY1101 | MMS | 0.0005% | 2.4 | 0.007 | 6 |
| SPY1102 | MMS | 0.0001% | 3.0 | 0.01 | 4 |
| SPY1103 | MMS | 0.0005% | 2.7 | 0.00009 | 6 |
| SPY1105 | MMS | 0.0001% | 2.2 | 0.004 | 4 |
| SPY1101 | γ-ray | 50 Gy | 4.8 | 0.004 | 2 |
| SPY1102 | γ-ray | 50 Gy | 3.9 | 0.01 | 2 |
| SPY1103 | γ-ray | 50 Gy | 3.1 | 0.01 | 2 |
| SPY1105 | γ-ray | 50 Gy | 2.8 | 0.0009 | 2 |

Loss of mag1 (mag1A) resulted in enhanced sensitivity to MMS and EMS. For example, after only four hours of exposure to MMS, SPY1102 were less viable than either SPY810 (wild-type control) or SPY1101 (control). However, SPY1102 showed no difference in viability when compared to either SPY810 or SPY1101 after four hours of γ-ray exposure. Moreover, the the MMS dose-dependent responses of SPY1101 and SPY1102 showed the enhanced sensitivity acquired through the MAG1 deletion. SPY1102 had a peak fluorescence of 154 fold at 0.005% MMS, whereas the optimum fluorescence of SPY1101 peak was 1.48 fold at 0.01%. At 0.005% MMS dose, SPY 1101 fluorescence was upreguiated 97 fold, indicating the MAG1 deletion caused a 1.5 fold increase in relative GFP expression at that dose. The MAG1 deletion had a more profound effect at the low MMS doses. SPY1101 had a relative fluorescence of 4 at 0.0005% MMS, whereas SPY1102 had a relative fluorescence of 34, an improvement of more than 8 fold. Thus, SPY1102 showed enhanced sensitivity to MMS, without altering its sensitivity to other tested genotoxins.

SPY1102 also showed increased sensitivity to EMS and 4-NQO compared to SPY1101.

Loss of MRE11 (SPY1103) resulted in an enhanced, sensitivity to CPT and γ-ray. Moreover, loss of MRE11 did not affect its ability to detect the other genotoxins. For example, after exposure, SPY1103 were less viable than SPY1101. However, SPY1103 showed no difference in viability when compared to SPY1101 when both were exposed to MMS. Moreover, the γ-ray dose-dependent responses of SPY1101 and SPY1103 showed the enhanced sensitivity acquired through the MRE11 deletion. Eight hours after exposure to γ-ray, SPY1103 had a greater fluorescence than that of SPY1101, which was the time point that resulted in the highest fluorescence induction (data not shown). The minimum detection threshold of SPY1101 was 200 Gy, with a relative fluorescence of 2.7. The same dose of γ-ray induced a relative fluorescence of 14.7 in mre11Δ, more than a 5 fold increase in signal over SPY1101. Meanwhile, SPY1103 were able to detect 50 Gy, with an 8-fold induction of fluorescence. Thus, SPY1103 showed enhanced sensitivity to γ-ray, without altering its sensitivity to other forms of DNA damage.

SPY1103 also showed increased sensitivity to CPT when compared to SPY1101.

Interestingly, SPY1105 showed additive effects from the two deletions, even though the SPY1103 phenotype (i.e., mre11Δ) predominated. For example, when SPY1105 were exposed to MMS, they displayed the SPY1103 phenotype. Although the sensitivity of SPY1105 to MMS was similar to that of SPY1102 (i.e., mag1Δ) with a peak fluorescence induction at 0.005%, the relative fluorescence at each dose was lower for SPY1105 than SPY1102. However, the relative fluorescence induction at 0.005% MMS was 49 fold for SPY1105, which was identical to the fluorescence induction of SPY1103 at its peak fluorescence. This induction was about 3-fold lower than the relative fluorescence induction of SPY1102. Basal GFP expression in SPY1105 was about 2.6 fold higher than that of SPY1101 when measured after culturing cells for 6 hours with no genotoxin present. The higher basal GFP expression in SPY1105 was most likely because of the gene deletions, which caused an increased accumulation of DNA damage from endogenous sources.

When SPY1105 were exposed to γ-ray, they also displayed an SPY1103 phenotype. Peak fluorescence for SPY1103 and SPY1105 occurred at 200 Gy and was not statistically different. Like SPY1102, the MAG1 deletion in SPY1105 had no effect on sensitivity to γ-rays

None of the deletion strains (e.g., SPY1102, SPY1103 or SPY1105) showed increased sensitivity to SDMH, HU, phleomycin and formaldehyde when compared to SPY1101 cells.

### Example 6

### (Prophetic) Using the Yeast-Based Biosensors to Discover Genotoxins

SPY1101, SPY1102, SPY1103 or SPY1105 are exposed to an agent suspected genotoxin, under conditions such as those described above in Examples 2 and 5. Control cells are the same cells, but are not exposed to the genotoxin.

If the agent suspected of being a genotoxin is genotoxic, one observes increased expression of the reporter gene relative to control. If the agent suspected of being a genotoxin is not genotoxic, one observes similar expression of the reporter gene relative to control.

### Example 7

### (Prophetic): Using the Yeast-Based Biosensors to Discover Genotoxin Inhibitors

SPY1101, SPY1102, SPY1103 or SPY1105 are exposed to a known genotoxin, under conditions such as those described above in Examples 2 and 5. However, the cells are simultaneously exposed to a putative inhibitor of the genotoxin being tested. Control cells are the same cells, exposed to the genotoxin but not to the putative inhibitor.

When exposed to a putative inhibitor that- blocks the action of the known genotoxin, one observes constant or reduced expression of the reporter gene relative to control. If the putative inhibitor does not block the action of the genotoxin, then one observes similar expression (i.e., within 5%) of the reporter gene relative to control.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

What is claimed is:

1. An expression vector comprising SEQ ID NO:1 operably linked to a reporter gene not natively linked to SEQ ID NO:1, wherein transcription of the reporter gene is increased in response to DNA damage caused by a genotoxin, but not in response to heat shock, starvation or formaldehyde.

2. The expression vector as recited in claim 1, wherein the reporter gene is selected from the group consisting of green fluorescent protein, yeast-enhanced green fluorescent protein, luciferase and β-galactosidase.

3. The expression vector as recited in claim 1, wherein the reporter gene is green fluorescent protein.

4. The expression vector as recited in claim 1, wherein the genotoxin is selected from the group consisting of alkylating agents, chemotherapeutic agents, DNA cross-linking agents, DNA topoisomerase inhibitors, oxidizing agents, ribonucleotide reductase inhibitors, UV light and ionizing radiation.

5. A yeast cell comprising an expression vector comprising SEQ ID NO:1 operably linked to a reporter gene not natively linked to SEQ ID NO:1, wherein transcription of the reporter gene is increased in response to DNA damage caused by a genotoxin, but not in response to heat shock, starvation or formaldehyde.

6. The yeast cell as recited in claim 5, wherein the reporter gene is selected from the group consisting of green fluorescent protein, yeast-enhanced green fluorescent protein, luciferase and β-galactosidase.

7. The yeast cell as recited in claim 5, wherein the reporter gene is green fluorescent protein.

8. The yeast cell as recited in claim 5, wherein the yeast cell is Saccharomyces cerevisiae.

9. The yeast as recited in claim 5, further lacking a gene selected from the group consisting of MAG1 and MRE11.

10. The yeast as recited in claim 5, further lacking MAG1 and MRE11.

11. A method of identifying a genotoxic agent, the method comprising the steps of:
exposing a yeast cell comprising an expression vector comprising SEQ ID NO:1 operably linked to a reporter gene not natively linked to SEQ ID NO:1 to an agent suspected of being a genotoxin, wherein transcription of the reporter gene is increased in response to DNA damage caused by a genotoxin, but not in response to heat shock, starvation or formaldehyde; and
measuring expression of the reporter gene, where genotoxicity of the agent correlates with increased expression of the reporter gene of host cells exposed to the agent relative to expression of the reporter gene of host cells not exposed to the agent.

12. The method as recited in claim 11, wherein the reporter gene is selected from the group consisting of green fluorescent protein, yeast-enhanced green fluorescent protein, luciferase and β-galactosidase.

13. The method as recited in claim 11, wherein the reporter gene is green fluorescent protein.

14. The method as recited in claim 11, wherein the yeast is Saccharomyces cerevisiae.

15. The method as recited in claim 11, wherein the yeast further lacks a gene selected from the group consisting of MAG1 and MRE11.

16. The method as recited in claim 11, wherein the yeast further lacks MAG1 and MRE11.

17. The method as recited in claim 11, wherein the genotoxin is selected from the group consisting of alkylating agents, chemotherapeutic agents, DNA cross-linking agents, DNA topoisomerase inhibitors, oxidizing agents, ribonucleotide reductase inhibitors, UV light and ionizing radiation.

18. A method of identifying a genoprotective agent, the method comprising the steps of:
exposing a yeast cell comprising an expression vector comprising SEQ ID NO:1 operably linked to a reporter gene not natively linked to SEQ ID NO:1 to an agent suspected of being a genotoxin and an agent suspected of being genoprotective, wherein transcription of the reporter gene is increased in response to DNA damage caused by a genotoxin, but not in response to heat shock, starvation or formaldehyde; and
measuring expression of the reporter gene of host cells exposed to both agents, where genoprotectivity of the agent vis-à-vis the genotoxic agent correlates with no increase or a decrease in expression of the reporter gene in host cells exposed to both agents relative to expression of the reporter gene of host cells exposed only to the genotoxic agent.

19. The method as recited in claim 18, wherein the reporter gene is selected from the group consisting of green fluorescent protein, yeast-enhanced green fluorescent protein, luciferase and β-galactosidase.

20. The method as recited in claim 18, wherein the reporter gene is green fluorescent protein.

21. The method as recited in claim 18, wherein the yeast is Saccharomyces cerevisiae.

22. The method as recited in claim 18, wherein the yeast further lacks a gene selected from the group consisting of MAG1 and MRE11.

23. The method as recited in claim 18, wherein the yeast further lacks MAG1 and MRE11.

24. The method as recited in claim 18, wherein the genotoxin is selected from the group consisting of alkylating agents, chemotherapeutic agents, DNA cross-linking agents, DNA topoisomerase inhibitors, oxidizing agents, ribonucleotide reductase inhibitors, UV light and ionizing radiation.
